(19) United States
(12) Patent Application Publication
(10) Pub. No.: US 2002/0128540 A1
(43) Pub. Date: Sep. 12, 2002

(54) System and method of correlating virtual reality with biofeedback for enhancing attention

(76) Inventors: Sun-Il Kim, Seoul (KR); Jan Han Lee, Anyang-si (KR); Dong Joo Kang, Seoul (KR); Dong-Pyo Jang, Sungnam-city (KR); In-Young Kim, Seoul (KR); Jae-Seok Kim, Seoul (KR); Back-Hwan Cho, Pusan (KR); Jeong-Hun Ku, Pusan (KR)

Correspondence Address: Welsh & Katz, Ltd., Thomas W. Tolpin, 22nd Floor, 120 South Riverside Plaza, Chicago, IL, 60606, US

(21) Appl. No.: 10/080,837

(22) Filed: Feb. 22, 2002

(30) Foreign Application Priority Data
Feb. 23, 2001 (KR) 10-2001-0009135

(51) Int. Cl. G06F 19/00; G01N 33/48; G01N 33/50; A61B 5/00

(52) U.S. Cl. 600/301; 702/19

(57) ABSTRACT

The present invention discloses a system and method for the enhancement of attention and further the treatment of attention deficit disorder. The present invention also presents a virtual reality environment for enhancing attention shift, selective attention, and sustained attention by correlating a biofeedback device that estimates the current attention level of the patient.

### FIELD OF THE INVENTION

The present invention relates to a system and method of enhancing attention and more particularly to a technique correlating virtual reality with biofeedback for enhancing attention.

### BACKGROUND OF THE INVENTION

Recent progress in politics, economics, and social environment has resulted in a variety of psychoses. The deficiency of attention can be thought of as a critical loss not only to the personal point of view but also to the society.

The attention is a fundamental recognizing capability, and the attention deficit disorder (ADD) results in difficulties in mental absorption, listening, and memorizing activities.

In other words, the psychiatric patients suffering from the attention deficit disorder (ADD) tend to become in attentive, impulsive, emotionally unstable, and sometimes excessively active.

The above-mentioned symptoms give rise to problems in studying, working, completing the responsibilities, and keeping company. The attention enhancement system is a therapy suffering from ADD (attention deficit disorder).

A conventional therapy for ADD is a drug-based treatment such as retalin. Another method for curing the ADD is a bio-graph employing a biofeedback.

The conventional treatments, however, have shortcomings in that the patient should visit a hospital or a therapy house in order to have a diagnosis and/or a treatment.

The virtual reality (VR) is a novel technology that enables the user to enter the computer-generated world and interact with computer through vision, auditory sense, and the sense of touch.

The virtual reality differs from the traditional displaying technique in a sense that it provides the user with the feeling of existence or the feeling of absorption as well as a variety of the computer graphic interface.

The virtual reality provides a paradigm where human being interacts with the computer, and consequently the user is no longer an observer that simply appreciates the computer image on the screen.

The user in virtual reality is absorbed in the activity in the three-dimensional virtual world that is generated by the computer.

The virtual environment is sometimes called in different terms such as virtual reality, remote reality, artificial world, cyber space, and multi-sense I/O (input and output).

The above-mention virtual reality can be employed for the treatment of ADD.

The children suffering from attention deficit disorder (ADD) have a tendency to respond very sensitively to the static and prompt compensation rather than the physical punishment.

The virtual reality enables the patient to feel the compensation from the computer-generated environment that is quite similar to reality. Due to absorbing property of the virtual reality, the virtual reality can enhance the chance of social adaptability of the patient.

### BRIEF SUMMARY OF THE INVENTION

The patient suffering from attention deficit disorder can be effectively treated through a virtual reality wherein the patient is not only provided with familiarity in practice and exercise in a computer-generated cyber classroom, which can be generalized in reality, but directly compensated by a cyber teacher via biofeedback.

Therefore, there is a need in the art for a therapy system and method for enhancing attention through virtual reality and biofeedback.

Accordingly it is an object of the present invention to provide a therapy system and method for enhancing attention as well as diagnosis of the attention level.

Yet it is another object of the present invention to provide a therapy system and method for enhancing attention that is adaptively applicable in accordance with the level of the patient's attention level.

It is also another object of the present invention to provide a therapy system and method for enhancing attention that correlates the treatment contents with the biofeedback.

A more detailed explanation of the invention is provided in the following description and appended claim taken in conjunction with the accompanying drawings.

### BRIEF DESCRIPTION OF THE DRAWINGS

FIG. 1 is a schematic diagram illustrating the configuration of a first embodiment of a system for enhancing attention in accordance with the present invention.

FIG. 2 is a schematic diagram illustrating the interaction between the biofeedback module and the virtual reality in accordance with the present invention.

FIG. 3 is a schematic diagram illustrating the configuration of a second embodiment of a system for enhancing attention in accordance with the present invention.

### DETAILED DESCRIPTION OF THE INVENTION

Preferred embodiments in accordance with the present invention will be explained in detail with reference to the accompanying drawings.

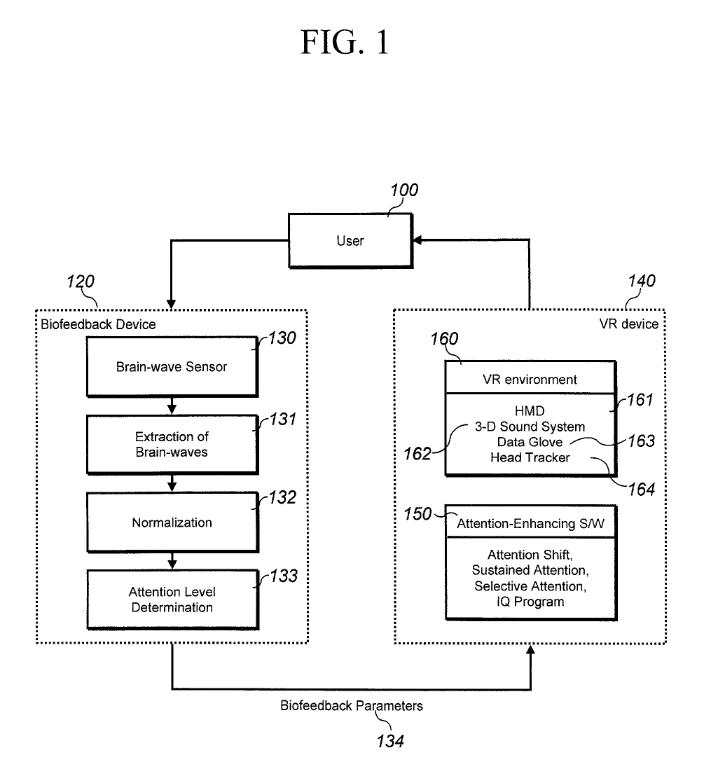

FIG. 1 is a schematic diagram illustrating a first preferred embodiment of a system for enhancing attention in accordance with the present invention. Referring to FIG. 1, the present invention includes a biofeedback device 120 and a virtual reality device 140.

The trainee or the patient is absorbed in a virtual reality computer 140 and the biofeedback device 120 detects the time-development of the trainee's brain wave. The data regarding the detected spectrum of the trainee's brain waves is then sent to a VR computer so that the VR computer 140 responds to the status of the trainee's attention status that is reflected on the biofeedback parameter.

In other words, the time-development of the brain wave, which correlates with the level of attention, is reflected on the new VR environment for the next-step exercise.

As a consequence, it becomes possible to evaluate the level of the trainee's attention and to provide the trainee with compensation for the response of the VR environment to the time change of the brain waves. The VR device 140 in accordance with the present invention comprises software 150 and hardware 160.

The hardware 160 comprising the VR device 140 includes an HMD (head mounted display) 161, a three-dimensional sound system 162, a data glove 163, and a head tracker 164.

The attention-enhancing system in accordance with the present invention comprises a variety of VR contents 150 for the diagnosis of the attention level as well as for the treatment.

As a preferred embodiment in accordance with the present invention, the VR contents comprise a visual comparison module, an auditory comparison module, a memory comparison module, and attention-shifting module.

The present invention has a feature that the system displays not only the level of attention but also the degree of improvement of the trainee's attention level. The VR contents in the present invention can comprise contents of attention shift, selective attention, attention concentration, and sustained attention.

The biofeedback device 120 in accordance with the present invention measures a spectrum of brain waves of the trainee 100 through a brain-wave sensor 130.

As a preferred embodiment of the brain wave sensor 130, either a sensor installed at the center of the head or the reference sensor installed at the edge of the ear can be employed.

The SMR-wave (12˜16 Hz), beta-wave (16˜20 Hz), and the theta-wave (4˜8 Hz) are extracted 131 from the data of the brain-wave sensor 130, and then sent to the VR device 140 after normalization 132.

As a preferred embodiment in accordance with the invention, the relative ratio of the strength of each brain-wave signal can be employed for the definition of the attention level.

More preferably, the brain-wave sensor 130 extracts a first beta-wave (13˜20 Hz), theta-wave (4˜8 Hz), alpha-wave (8˜13 Hz), and a second beta-wave (20˜40 Hz).

Preferably, the level of attention can be evaluated by calculating the ratio of the sum of a first beta-wave and a second beta-wave to the sum of theta-wave and alpha-wave.

In this case, a variety of VR contents can be classified in such a way that they match each level of attention. As another preferred embodiment of the present invention, the duration of performance of each VR content can be controlled in accordance with the trainee's attention level.

In addition, the attention level of the trainee is initially determined and then the appropriate level of VR contents can be chosen for the trainee's exercise.

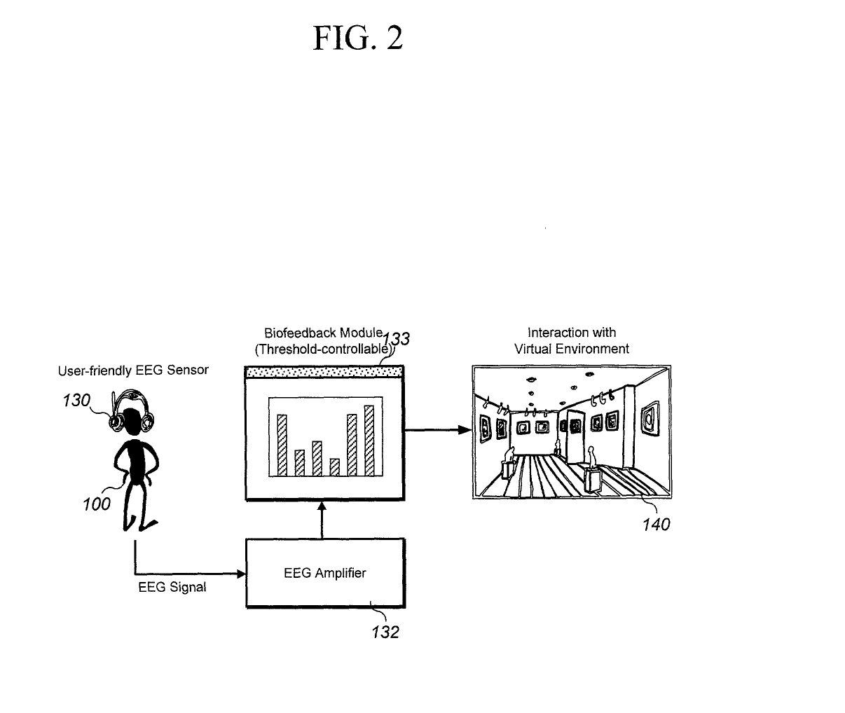

FIG. 2 is a schematic diagram illustrating the interaction between the biofeedback module and the virtual reality in accordance with the present invention.

Referring to FIG. 2, the brain wave is detected through the brain-wave detector 130 of the trainee 100 and then normalized at EEG amplifier 132.

The relative ratio of the strength of each brain wave is thereafter calculated at a biofeedback module 133 to evaluate the level of the trainee's attention.

Now, the VR contents for attention enhancement are selected in accordance with the current level of the trainee's attention. As an exemplary preferred embodiment, the VR environment can be assumed to be a classroom for students.

At the front of the classroom is located a large screen. A teacher is now explaining the subjects while there is a large desk in front of the trainee. The trainee takes a lot of subjects and gives answers for attention enhancement on the desk.

Preferably, the subjects for attention enhancement include visual comparison module, auditory comparison module, memory comparison module, and attention shifting module.

As a preferred embodiment of visual comparison module in accordance with the present invention, a couple of objects out of a circular cylinder, a circular cone, a sphere, a quadrilateral cone, a triangular cone, a regular hexahedron, a hexahedron can be chosen to show up on the desk.

After comparing the two objects that are shown in front of him, the trainee clicks the mouse if he thinks the two objects are identical. Furthermore, the next question asking if he thinks that the currently displayed objects of the two are identical can be presented after some interval.

Preferably, the level of difficulty can be adjusted by employing objects with wide range of complexity. The biofeedback VR system in accordance with the present invention presents VR contents comprising a couple of geometric objects to the trainee.

The trainee is then requested to answer to the questions related to the objects. Now, the VR contents are updated with different visual element for presentation in response to the trainee's answer.

As a preferred embodiment of auditory comparison module in accordance with the invention, a vocabulary can listed in a moving fashion on the monitor while a teacher is explaining about a subject that is correlated with the current vocabulary.

In this case, the trainee is supposed to click the mouse once he thinks the listed vocabulary coincides with the teacher's explanation or the implication of the environment.

Now, the response time of the trainee's recognition to the abrupt appearance of a vocabulary on the monitor can be evaluated and then the level of attention is calculated with the evaluated response speed.

In response to the trainee's attention level, a subsequent set of problems asking the trainee if the associated word is supposed to have something to do with what he listens.

Then the VR computer can either change the displayed moving speed of the associated word or alter the auditory elements of the VR contents. As a consequence, the attention level of the trainee can be forced to enhancement.

As a preferred embodiment of memory comparison module in accordance with the present invention, the trainee can be asked to detect the change in the classroom environment where a new item is stealthily introduced in the VR environment under fade-in and fade-out mode.

The above-mentioned embodiment can be employed for enhancing the environment memorizing capability of the trainee. In other words, a first VR content that has been chosen in response to the attention level of the trainee is made gradually disappear and replaced by a second VR content. In this case, there is a minor change between the first and second VR contents for testing the trainee's recognition capability.

Then the trainee has to detect an object in the current VR content from the previous VR content.

Now, the VR computer updates the level of the contents for the trainee' exercise in response to the trainee's answer to the inquiry of telling the difference between a couple of successive VR contents.

Additionally, as a preferred embodiment of attention shifting module in accordance with the present invention, the VR computer can present several models like a star, a ball, or a bell together with a reference model.

In this case, the trainee is requested to choose one model that he believes to be the most similar to the reference model in terms of shape, coloring, or other features.

Preferably, the VR computer can respond to the trainee's answer by generating a specific sound for correct answer.

More preferably, the trainee can be asked to choose a second most similar object by having him reconsider other features of the reference model if he has selected a wrong answer.

In other words, the present invention presents a multiple of object models with the reference model for comparison. Then the trainee is requested to choose the most appropriate one out of a multiple of object models that he thinks to be the most similar to the reference model.

If the trainee's answer is correct, the VR computer upgrades the level of attention-shift capability in order to induce the enhancement of the trainee's attention.

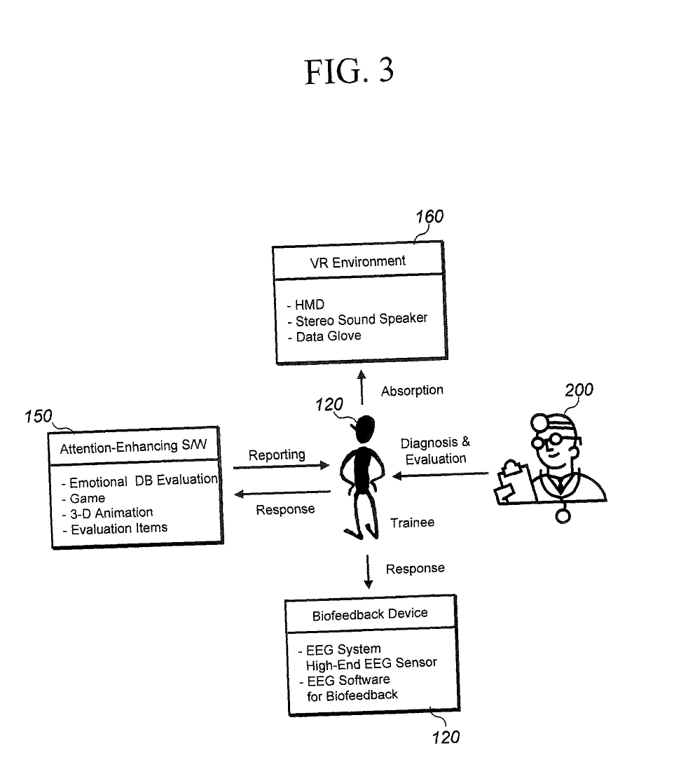

FIG. 3 is a schematic diagram illustrating a second embodiment of a VR system in accordance with the present invention. Referring to FIG. 3, the VR system for enhancing attention comprises a virtual reality environment 160, an attention enhancement software module 150, a biofeedback device 120, and a doctor or a trainer 200.

The biofeedback device 120 in accordance with the invention senses brain waves of specific spectral ranges including a first beta-wave (13˜20 Hz), a second beta-wave (20˜40 Hz), an alpha-wave (8˜13 Hz), and a theta-wave (4˜8 Hz).

Now, the attention level of the trainee can be evaluated by estimating the strength ratio of the sum of the first and second beta-waves and the sum of the alpha-wave and the theta-wave.

Preferably, the level of the VR contents can be downgraded if the attention level of the trainee goes up. In the meanwhile, the level of the VR contents can be upgraded if the attention level of the trainee goes downward.

More preferably, if the trainee's attention level goes downward, the speed of the moving object in the VR contents should be raised.

In addition, if the trainee's attention level goes upward, the VR contents wherein the velocity of the moving object is reduced are preferably provided to the trainee in order to enhance the trainee's attention level.

More preferably, if the trainee's attention level goes downward, the VR contents with relative difficulty can be presented for the enhancement of the trainee's attention.

In the meanwhile, the updated VR contents should include relatively less difficult exercises if the trainee's attention level goes up.

As a preferred embodiment in accordance with the present invention, the trainee 100 is absorbed in the VR while the trainee's attention level is estimated from the information of the detected brain waves through the biofeedback sensor.

Thereafter, the VR contents are updated in response to the estimated level of the trainee's attention. Now, the doctor 200 can make diagnosis of the trainee 100 both in reality and in virtual reality.

Although the invention has been illustrated and described with respect to exemplary embodiments thereof, it should be understood by those skilled in the art that various other changes, omissions and additions may be made therein and thereto, without departing from the spirit and scope of the present invention.

Therefore, the present invention should not be understood as limited to the specific embodiment set forth above but to include all possible embodiments which can be embodies within a scope encompassed and equivalents thereof with respect to the feature set forth in the appended claims.

What is claimed is:

1. A method of enhancing the attention of a trainee interacting with a computer-generated virtual reality, comprising steps of:
(a) extracting and normalizing brain waves from said trainee through a biofeedback sensor;
(b) estimating the attention level of the trainee by calculating the relative ratio of the strength level of each brain wave; and
(c) updating the visual and/or auditory elements in the VR contents in response to the estimated attention level of the trainee for improving the trainee's attention.

2. The method as set forth in claim 1 wherein said step of (a) comprises a step of extracting and normalizing beta-wave of 13˜20 Hz, theta-wave of 4˜8 Hz, and SMR-wave of 12˜16 Hz.

3. The method as set forth in claim 1 wherein said VR contents comprise either one or the combination from a group of visual comparison module, auditory comparison module, memory comparison module, and attention shifting module.

4. The method as set forth in claim 1 wherein said step of (c) comprises steps of:
(d) extracting beta-wave of 13˜20 Hz;
(e) extracting theta-wave of 4˜8 Hz;
(f) determining the current attention level of the trainee by calculating the relative ratio of the strength level between said beta-wave and theta-wave; and
(g) updating a VR content in response to the calculated attention level of the trainee.

5. The method as set forth in claim 1 wherein said step of (c) comprises steps of:
(d) displaying a couple of objects in the VR content in response to the trainee's attention level;
(e) having the trainee to give an answer to a question asking the trainee to tell the difference of geometric feature between the two objects; and
(f) presenting an updated VR content with an altered visual element for changing the difficulty level in response to the evaluation about said trainee's answer to the previous question.

6. The method as set forth in claim 1 wherein said step of (c) comprises steps of:
(d) displaying a vocabulary that is listed in a moving fashion on the screen while asking the trainee if he thinks said listed vocabulary has something to do with what he listens to in VR environment;
(e) having the trainee to respond to the inquiry of step (d) by clicking a mouse or typing a keyboard; and
(f) inducing the enhancement of the trainee's attention by altering the moving speed of said listed vocabulary or changing the auditory elements of said VR content in accordance with the trainee's response at step (e).

7. The method as set forth in claim 1 wherein said step of (c) comprises steps of:
(d) presenting the trainee with a first VR content in accordance with the estimated level of the trainee's attention;
(e) having said first VR content faded gradually away and a second VR content faded gradually in;
(f) having the trainee to compare the first VR content and the second VR content and to find the object that has stealthily showed up in the second VR content; and
(g) inducing the enhancement of the trainee's attention by presenting a new VR content that has been leveled up or down in accordance with the trainee's response at step (f), respectively.

8. The method as set forth in claim 1 wherein said step of (c) comprises steps of:
(d) presenting a multiple of object models with a reference model to the trainee in accordance with the trainee's current level of attention;
(e) having the trainee to designate one out of said multiple of object models which he thinks is to be the most similar to the reference object model; and
(f) inducing the enhancement of the trainee's attention by presenting a new VR content that has been leveled up or down in accordance with the trainee's response at step
(e), respectively.

9. The method as set forth in claim 1 wherein said step of (c) comprises steps of:
(d) extracting a first beta-wave of 13˜20 Hz;
(e) extracting a second beta-wave of 20˜40 Hz;
(f) extracting an alpha-wave of 8˜13 Hz;
(g) extracting a theta-wave of 4˜8 Hz;
(h) evaluating the current level of the trainee's attention by calculating the ratio between the sum of the first beta-wave and the second beta-wave and the sum of the alpha-wave and the theta-wave; and
(i) presenting a new VR content that has been leveled up if the level of the trainee's attention level drops and leveled down if the trainee's attention level rises.

10. The method as set forth in claim 1 wherein said step of (c) comprises a step of:
presenting a new VR content where the moving speed of an object has been raised if the attention level of the trainee drops downward and vice versa.

11. The method as set forth in claim 1 where said step of (c) comprises a step of:
presenting an updated VR content with higher difficulty level if the attention level of the trainee drops downward and vice versa.

12. A system for enhancing attention or treating attention deficit disorder of a trainee, comprising:
a biofeedback device producing a multiple of signals from the extraction of the trainee's brain waves of specific frequency ranges;
a processor that estimates the trainee's attention level by calculating the strength ratio of each signal from the normalized brain waves for each frequency range; and
a virtual reality device having a variety of VR contents and presenting the trainee with an updated VR content for attention enhancement in accordance with the currently estimated attention level of the trainee.

13. The system as set forth in claim 12 wherein said VR device comprises:
a head mounted device (HMD);
a head tracker;
a sound speaker; and
a variety of VR contents with different levels of difficulty.

14. The system as set forth in claim 12 wherein said processor comprises a unit that estimates the current attention level of the trainee and controls a command to present a VR content with updated level of difficulty in accordance with the estimated current attention level of the trainee.

15. The system as set forth in claim 12 wherein said biofeedback device extracts either one or the combination from the group of beta-wave of 13˜20 Hz, theta-wave of 4˜8 Hz, and alpha-wave of 8˜13 Hz.

16. The system as set forth in claim 12 wherein said biofeedback device extracts either one or the combination from the group of a first beta-wave of 13˜20 Hz, a second beta-wave of 20˜40 Hz, a theta-wave of 4˜8 Hz, and an alpha-wave of 8˜13 Hz.

17. The system as set forth in claim 12 wherein said VR device comprises VR contents including one or the combination from the group of visual comparison module, auditory comparison module, memory comparison module, and attention shifting module.

18. The system as set forth in claim 12 wherein said VR device comprises VR contents for one or the combination from the group of attention shift, selective attention, attention, and sustained attention.
